(19) United States
(12) Patent Application Publication
(10) Pub. No.: US 2025/0118401 A1
(43) Pub. Date: Apr. 10, 2025

(54) GENERATING EMBEDDINGS OF MEDICAL ENCOUNTER FEATURES USING SELF-ATTENTION NEURAL NETWORKS

(71) Applicant: Google LLC, Mountain View, CA (US)

(72) Inventors: Edward Choi, San Jose, CA (US); Andrew M. Dai, San Francisco, CA (US); Gerardo Flores, Berkeley, CA (US); Yuan Xue, Palo Alto, CA (US); Michael Ward Dusenberry, San Mateo, CA (US); Zhen Xu, Mountain View, CA (US); Yujia Li, London (GB)

(21) Appl. No.: 17/143,083

(22) Filed: Jan. 6, 2021

Related U.S. Application Data
(60) Provisional application No. 62/957,682, filed on Jan. 6, 2020.

(51) Int. Cl. G16H 15/00; G06N 3/08; G16H 50/20

(52) U.S. Cl. CPC G16H 15/00; G06N 3/08; G16H 50/20

(57) ABSTRACT

Methods, systems, and apparatus, including computer programs encoded on computer storage media, for processing data about a medical encounter using neural networks. One of the methods includes obtaining features for a medical encounter associated with the patient, each feature representing a corresponding health event associated with the medical encounter and each of the plurality of features belonging to a vocabulary of possible features that each represent a different health event; and generating respective final embeddings for each of the features for the medical encounter by applying a sequence of one or more self-attention blocks to the features for the medical encounter, wherein each of the one or more self-attention blocks receives a respective block input for each of the features and applies self-attention over the block inputs to generate a respective block output for each of the features.

### CROSS-REFERENCE TO RELATED APPLICATION

This application claims priority to U.S. Provisional Application No. 62/957,682, filed on Jan. 6, 2020. The disclosure of the prior application is considered part of and is incorporated by reference in the disclosure of this application.

### BACKGROUND

This specification relates to processing electronic health record data using a neural network.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters.

### SUMMARY

This specification describes a system of one or more computers in one or more physical locations that generates embeddings of features of a medical encounter associated with a patient.

Particular embodiments of the subject matter described in this specification can be implemented so as to realize one or more of the following advantages.

Electronic health record (EHR) data, i.e., data derived from the electronic health record of a patient, for a given medical encounter has an underlying graphical structure (e.g., has relationships between diagnoses and treatments). However, the EHR data for any given encounter does not always contain complete structure information that identifies this structure. In fact, in some cases, this structure may not be available at all. For example, the EHR data may include multiple diagnoses and a treatment, without any indication of which diagnosis or diagnoses lead to the treatment being given to the patient. Similar issues can exist with insurance claims data, which may only identify the health events that occurred during an encounter without any indication of which of those health events are related.

Under such circumstances, the techniques described in this specification can leverage the implicit structure of the encounter data by making use of self-attention. The use of self-attention as described in this specification can improve the quality of the representation of the medical encounter, i.e., by allowing the system to generate embeddings that can be used to generate accurate health predictions without making use of explicit structure information.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A shows an example of the graphical structure of a medical encounter.

FIG. 1B shows an example feature embedding system.

FIG. 2 is a flow diagram of an example process for generating embedding of features of a medical encounter.

FIG. 3 is a flow diagram of an example process for modifying the operations performed by the self-attention neural network.

Like reference numbers and designations in the various drawings indicate like elements.

### DETAILED DESCRIPTION

This specification describes a system of one or more computers in one or more physical locations that generates embeddings of features of a medical encounter associated with a patient. Generally, the medical encounter is an interaction between the patient and one or more medical professionals and the features represent health events occurring during the encounter.

Examples of medical encounters include visits or consultations with a doctor and an admission to a medical facility for treatment. The features can include features generated from diagnoses made by a medical professional, features generated from treatments given to the patient, features generated from lab results, and so on. The features can be obtained from electronic health record data for the patient.

An embedding, as used in this specification, is a numeric representation of a medical event. In particular, an embedding is a numeric representation in an embedding space, i.e., an ordered collection of a fixed number of numeric values, where the number of numeric values is equal to the dimensionality of the embedding space. For example, the embedding can be a vector of floating point or other type of numeric values that has a certain dimensionality.

Once the embeddings have been generated, the system can use the embeddings to generate a prediction that characterizes the health of the patient.

For example, the system can generate, from the respective embeddings of the features, a representation of the medical encounter and process an input including the representation of the medical encounter using a downstream neural network to generate a prediction that characterizes the health of the patient. In some cases, this input also includes representations of previous medical encounters associated with the patient.

As another example, the system can use the embeddings to predict the structure of the features. For example, the system can compute an inner-product between each feature embedding pair and then determine that two features are connected within the health record data when the inner-product for the corresponding feature embedding pair satisfies a threshold. For example, the system can determine, based on the inner-product between a diagnosis feature and a treatment feature satisfying the threshold, that the diagnosis lead to the treatment being prescribed. As another example, the system can determine, based on the inner-product between a treatment feature and a lab result feature satisfying the threshold, that the treatment lead to the lab result being obtained.

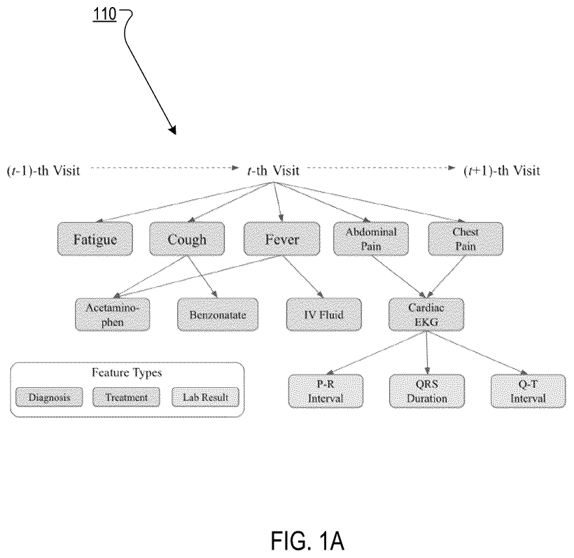

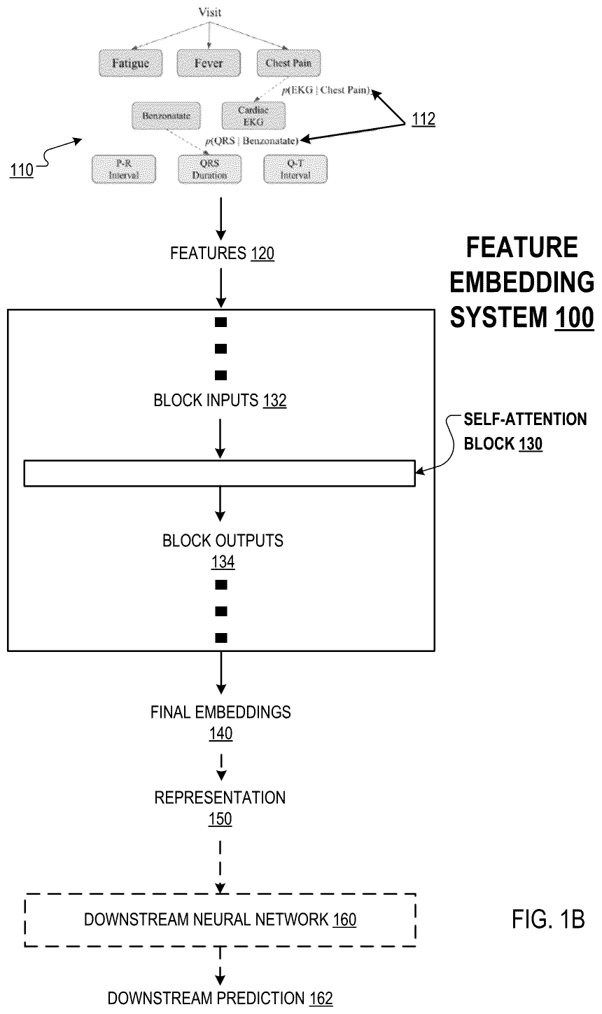

FIG. 1A shows an example of the graphical structure of an example medical encounter 110. The medical encounter 110 is a visit at time t by a patient to a medical professional. During the medical encounter 110 three different types of health events occurred: diagnoses, treatments, and lab results. In more detail, during the medical encounter 110, five diagnoses are made: that the patient has “fatigue,” that the patient has a “cough,” that the patient has a “fever,” that the patient has “abdominal pain,” and that the patient has “chest pain.” These five diagnoses (and, in some cases, other information) result in four treatments: “Acetaminophen,” “Benzonatate,” “IV Fluid,” and a “Cardiac EKG.” As a result of the “Cardiac EKG” being prescribed, three lab results were obtained: “P-R Interval,” “QRS Duration,” and “Q-T Interval.”

As can be seen from the example in FIG. 1A, the various health events occurring during the encounter 110 have a graphical structure that is represented in the Figure using arrows. In particular the arrows represent edges in a graph representation of the encounter 110. For example, the IV Fluid treatment was prescribed because of the Fever diagnosis, but not because of the chest pain diagnosis and is therefore connected by an arrow to the fever diagnosis, but not the chest pain diagnosis. As another example, the three lab results were obtained as a result of the Cardiac EKG treatment, and therefore are connected by arrows to the Cardiac EKG treatment and not any of the three other treatments that were prescribed during the encounter 110.

This graphical structure can be very informative for making predictions that relate to the health of the patient, e.g., for predicting the likelihood of future adverse health events occurring or for predicting additional treatments that may benefit the patient. However, the graphical structure is missing from the data that is received by the prediction system that and that documents the encounter 110.

For example, EHR data may not have reliable or complete links between diagnoses and the treatments that resulted from the diagnoses. For example, some datasets might describe which treatment lead to measuring certain lab values, but might not describe the reason for ordering that treatment, i.e., the diagnosis that resulted in the treatment.

As another example, insurance claim data may only record health events occurring during the encounter 110 without any connection between the health events.

By processing features of a medical encounter using the techniques described in this specification to generate embeddings of the features, the resulting embedding can better reflect dependencies between health events occurring in the medical encounter and, therefore, result in improved predictions about the health of the patient involved in the medical encounter.

FIG. 1B shows an example feature embedding system 100. The feature embedding system 100 is an example of a system implemented as computer programs on one or more computers in one or more locations in which the systems, components, and techniques described below are implemented.

The system 100 generates final embeddings 140 of features 120 of a medical encounter 110 associated with a patient.

An embedding, as used in this specification, is a numeric representation of a medical event. In particular, an embedding is a numeric representation in an embedding space, i.e., an ordered collection of a fixed number of numeric values, where the number of numeric values is equal to the dimensionality of the embedding space. For example, the embedding can be a vector of floating point or other type of numeric values that has a certain dimensionality.

Generally, the medical encounter 110 is an interaction between the patient and one or more medical professionals and the features represent health events occurring during the encounter.

Examples of medical encounters include visits or consultations with a doctor and an admission to a medical facility for treatment.

The features 120 can include features generated from diagnoses made by a medical professional, features generated from treatments given to the patient, features generated from lab results, and so on.

Generally, the features 120 are received as input by the system 100 and are numeric representations of the corresponding health event in a latent space of fixed dimensionality. The features 120 can be obtained from or generated from electronic health record data for the patient that describes the corresponding health event. Examples of features include features generated from categorical or continuous representations of diagnostic codes, lab values, treatments, and so on, e.g., by embedding the categorical representations in the latent space or by projecting the continuous representations into the latent space.

Once the final embeddings 140 have been generated, the system 100 can provide the embeddings 140 to another system or use the embeddings 140 to generate a prediction, e.g., a downstream prediction 162, that characterizes the health of the patient.

For example, the system 100 can generate, from the respective embeddings 140 of the features, a representation 132 of the medical encounter and process an input including the representation 132 of the medical encounter using a downstream neural network 160 to generate a prediction 162 that characterizes the health of the patient.

The system can generate the representation 132 of the medical encounter 110 in any of a variety of ways.

As one example, the features 120 can include a placeholder feature, i.e., a feature that has a predetermined default value, representing the encounter 110 and, therefore, the final embeddings 140 will include a final embedding for the placeholder feature. In this example, the representation 132 of the medical encounter 110 can be the final embedding for the placeholder feature representing the encounter 110.

As another example, the system 100 can generate the representation 132 of the medical encounter 110 by aggregating the respective final embeddings 140, e.g., by averaging the final embeddings 140 or applying a pooling operation to the final embeddings 140.

In some cases, this input also includes representations of previous medical encounters associated with the patient.

The downstream prediction 162 can include, e.g., a prediction of a final diagnosis for the patient resulting from the encounter 110.

As another example, the prediction 162 can include a prediction of the likelihood of an adverse health event occurring to the patient after the encounter, e.g., within a certain time window of the encounter. Adverse health events can include acute kidney injuries, heart failure, sepsis, a patient health deterioration event, an abnormal physiological sign, readmission to a medical care facility, a discharge from a medical care facility (i.e., a likelihood that the patient will be unsafely discharged), an admission to an intensive care unit (ICU), mortality, and so on.

As another example, the system 100 can use the embeddings 140 to predict the structure of the features 120. For example, the system 100 can compute an inner-product between each feature embedding pair, i.e., each pair of embeddings 140, and then determine that two features 120 are connected within the health record data when the inner-product for the corresponding feature embedding pair satisfies a threshold. For example, the system can determine, based on the inner-product between a diagnosis feature and a treatment feature satisfying the threshold, that the diagnosis lead to the treatment being prescribed. As another example, the system can determine, based on the inner-product between a treatment feature and a lab result feature satisfying the threshold, that the treatment lead to the lab result being obtained. Thus, in this case, the prediction 162 includes a prediction for each of one or more pairs of features that indicates a likelihood that the features are connected in a ground truth graph representation of the encounter 110.

The system generates the final embeddings 140 by processing the features 120 using a self-attention neural network that applies a sequence of one or more self-attention blocks 130 to the features 120 for the medical encounter.

Each of the one or more self-attention blocks 130 receives a respective block input 132 for each of the features 120 and applies self-attention over the block inputs 132 to generate a respective block output 134 for each of the features 120. In other words, the input to each self-attention block 130 is a respective block input for each of the features 120 that has the same dimensionality as the feature and each self-attention block 130 updates the block inputs to generate a respective block output for each of the features 120 that is the same dimensionality as the feature.

The respective block inputs 132 for the first self-attention block 130 in the sequence are the features 120 for the medical encounter 110. The respective block inputs 132 for each block after the first self-attention block 130 in the sequence are the features 120 for the medical encounter 110.

The block outputs of the last self-attention block in the sequence are the respective final embeddings 140 for the features 120.

In some implementations, to generate block outputs from block inputs, each self-attention block generates, from the block inputs, a respective query for each feature by applying a first, learned linear transformation to the block input for the feature, a respective key for each feature by applying a second, learned linear transformation to the block input for the feature, and a respective value for each feature by applying a third, learned linear transformation to the block input for the feature.

For each particular feature, the self-attention block then generates the output of the self-attention for the particular feature as a linear combination of the values for the features, with the weights in the linear combination being determined based on a similarity between the query for the particular feature and the keys for the features.

In particular, in some implementations, the operations for the self-attention mechanism for a given self-attention block can be expressed as follows:

$$C = soft max ( \frac{{QK}^{T}}{\sqrt{d}} ) V ,$$

where C is a matrix that includes a respective self-attention output for each of the features, Q is a matrix of the queries for the features, K is a matrix of the keys for the features, Vis a matrix of the values of the features, and dis a scaling factor, e.g., equal to the dimensionality of the features or of the queries, keys, and values. Thus,

$$soft max ( \frac{{QK}^{T}}{\sqrt{d}} )$$

is a matrix of the attention weights for the self-attention block.

In some cases, the output of the self-attention mechanism is the block outputs of the self-attention block. In some other cases, the self-attention block can perform additional operations on the output of the self-attention mechanism to generate the block outputs for the block, e.g., by applying one or more of residual connections, feed-forward layer operations, and layer normalization operations to the outputs of the-self-attention mechanism.

The above description of self-attention describes un-masked self-attention, where any attention weight for any given feature can take a non-zero value.

In some implementations, however, one or more of the self-attention blocks applies masked self-attention, in which attention weights are modified using a masking in which one or more of the attention weights are constrained to have a zero value. In particular, in some implementations, a particular self-attention block can apply masking based on vocabulary data that specifies connections between features in the vocabulary of possible features, i.e., so that the attention weight between any two features is constrained to be zero if no connection between the two features is specified in the vocabulary data.

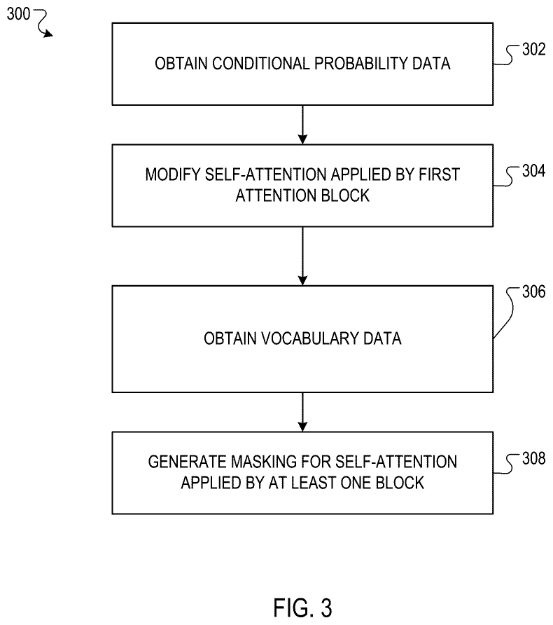

Masked self-attention is described in more detail below with reference to FIG. 3.

Additionally, in some other implementations, the system 100 uses conditional probabilities 112 to modify the self-attention that is applied by the first self-attention block, i.e., to the features 120 for the medical encounter 112. The conditional probabilities 112 specify, for each of one or more pairs of features, conditional probabilities of the first feature in the pair occurring in a particular encounter given that the second feature in the pair occurred in the particular encounter, e.g., as determined from historical or aggregate health record data for a large number of patients.

Using the conditional probabilities 112 to modify the operations performed by first self-attention block in the sequence is described below with reference to FIG. 3.

Thus, by repeatedly applying self-attention to the features 120 to generate the final embeddings 140, the system 100 generates embeddings that, for any given feature, reflect not only the characteristics of the feature itself, but also the connectivity of the feature to the other features for other health events that occurred during the encounter 110, even when that information is not present in the input received by the system 100.

Generally, the system 100 trains the self-attention neural network to minimize a loss function for the prediction task that the embeddings 140 are used to perform after being generated by the self-attention neural network. That is, the system 100 trains the self-attention neural network and any operations that are used to generate the prediction 162, e.g., jointly with the downstream neural network 160, jointly on training data that includes multiple training examples, with each training example including (i) features of an encounter and (ii) a ground truth prediction that should be generated for the encounter.

In some implementations, the system 100 also adds additional loss terms to the loss function that regularize the training of the self-attention neural network. These additional loss terms are described in more detail below with reference to FIG. 3.

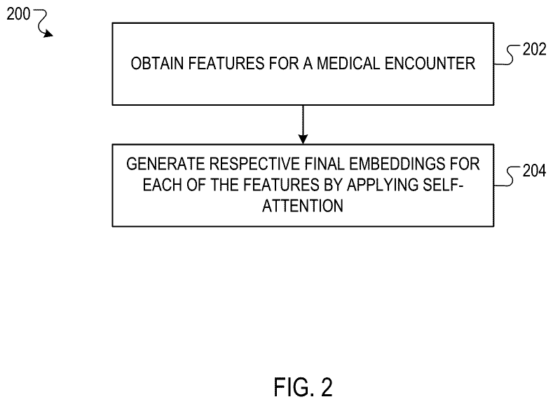

FIG. 2 is a flow diagram of an example process 200 for generating embeddings of features of a medical encounter. For convenience, the process 200 will be described as being performed by a system of one or more computers located in one or more locations. For example, a feature embedding system, e.g., the feature embedding system 100 of FIG. 1B, appropriately programmed, can perform the process 200.

The system obtains features for a medical encounter associated with a patient (step 202). For example, the system can receive features that have been generated from information in electronic health record data for the patient or from insurance claims data for the medical encounter. Each feature represents a corresponding health event associated with the medical encounter and each of the plurality of features belongs to a vocabulary of possible features that each represent a different health event.

The system generates respective final embeddings for each of the features for the medical encounter by applying a sequence of one or more self-attention blocks to the features for the medical encounter (step 204). As described above, each of the one or more self-attention blocks receives a respective block input for each of the features and applies self-attention over the block inputs as part of generating a respective block output for each of the features. The final embeddings are then the block outputs generated by the final self-attention block in the sequence.

As described above, in some implementations, the system modifies the self-attention performed by one or more of the blocks using conditional probabilities, masking, or both. This is described in more detail below.

FIG. 3 is a flow diagram of an example process 300 for modifying the operations performed by the self-attention neural network. For convenience, the process 300 will be described as being performed by a system of one or more computers located in one or more locations. For example, a feature embedding system, e.g., the feature embedding system 100 of FIG. 1A, appropriately programmed, can perform the process 300.

The system obtains conditional probability data (step 302). The conditional probability data specifies conditional probabilities for certain pairs of features from the vocabulary of features. In particular, for a given first feature-second feature pair, the conditional probability is a probability of the first feature occurring in a particular encounter given that the second feature occurred in the particular encounter. The conditional probability data can be generated from a set of encounters identified in a data set of health record data for a large number of patients, with the conditional probability for a given first feature-second feature pair being computed as (i) the total number of encounters in the data set for which both the first feature and the second feature occurred divided by (ii) the total number of encounters in the data set for which the second feature occurred.

The system modifies the operation of the first self-attention block in the sequence using the conditional probability data (step 304). In particular, the first self-attention layer applies attention weights that are generated based on the conditional probability data rather than on a similarity between the queries and the keys. In particular, the self-attention output C generated by the first self-attention block can satisfy:

C=PV,
where, as above, V is the matrix of values for the first self-attention block, and P is a matrix of conditional probability values for pairs of features that represent health events that occurred in the encounter. In some cases, the conditional probabilities in P are normalized so that the rows of P sum to one.

Thus, in these cases, the first attention block applies self-attention that is based on prior information regarding which features are more likely to be connected to which other features.

The system obtains vocabulary data that specifies connections between features in the vocabulary of possible features (step 306). That is, the vocabulary data specifies, for each feature in the vocabulary, which other features the feature can be connected to. For example, the vocabulary data can specify that treatment features are only connected to diagnosis features, but not to other treatment features.

The system generates mask data that modifies the self-attention that is applied by at least one of the attention blocks such that the self-attention is masked self-attention that decreases, for each particular feature, the impact of block inputs for features that are not connected to the particular feature in the vocabulary data on the block output for the particular feature (step 308).

For example, the self-attention applied by each block after the first block in the sequence can be masked using the vocabulary data.

In some implementations, the masking is a “hard” masking that assigns an attention weight of zero to features that are not connected to the particular feature in the vocabulary data when computing the block output for the particular feature.

In some other implementations, the system uses a combination of “normal” attention weights and “masked” attention weights to generate the final attention weights. In particular, the system can generate a mask matrix M that has negative infinities where connections are not allowed, and zeros where connections are allowed. The output of the masked self-attention can then satisfy:

$$C = soft max ( \frac{{QK}^{T}}{\sqrt{d}} + M ) V .$$

As described above, in implementations where conditional probabilities and masking are used, the system can include one or more additional terms that regularize the training of the self-attention neural network. In particular, the system can include additional terms that regularize the training to penalize attention weights of each self-attention block from deviating from attention weights generated by the preceding self-attention block. For example, for each layer, there can be an additional term that is equal to or proportional to the KL divergence between (i) the attention weights generated by the preceding layer and (ii) the attention weights generated by the layer. When masked self-attention is used, the KL divergence can be between masked attention weights (equal to

$$soft max ( \frac{{QK}^{T}}{\sqrt{d}} + M ) )$$

for the respective self-attention layers. When conditional probabilities are used, the system can calculate a set of masked attention weights for the first layer as described above for use in computing the KL divergence for the second layer. For the first layer, the auxiliary term can be a KL divergence between the matrix P and the masked attention weights for the first layer.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs. The one or more computer programs can comprise one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more computers, the method comprising:
obtaining, by the one or more computers, a plurality of features from one or more records associated with an individual, wherein each feature represents a corresponding event indicated by the one or more records and each of the plurality of features belongs to a vocabulary of multiple features that each represent a different event, and wherein relationships exist among at least some of the events indicated by the one or more records and the one or more records omit one or more of the relationships among the events;
maintaining, by the one or more computers, vocabulary data that specifies, for each of the features in the vocabulary, respective connections between the feature and one or more other features in the vocabulary;
generating, by the one or more computers, respective embeddings for each of the features from the one or more records by applying a self-attention neural network that comprises a sequence of one or more self-attention blocks to the features from the one or more records, wherein each of the one or more self-attention blocks receives a respective block input for each of the features and applies self-attention over the block inputs to generate a respective block output for each of the features, and wherein, for one or more of the self-attention blocks, applying self-attention over the block inputs comprises:
generating, from the block inputs, a respective query for each feature by applying a first, learned linear transformation to the block input for the feature;
generating a respective key for each feature by applying a second, learned linear transformation to the block input for the feature;
generating a respective value for each feature by applying a third, learned linear transformation to the block input for the feature;
determining, for each feature, a respective attention weight for each of the plurality of features, wherein:
(i) for each other feature that is not connected to the feature in the vocabulary data, the respective attention weight is based on a respective similarity between the query for the feature and the key for the other feature and on a mask value that decreases an impact of the other feature on the feature, and
(ii) for each other feature that is connected to the feature in the vocabulary, the respective attention weight is based on a respective similarity between the query for the feature and the key for the other feature; and
combining, for each feature, the respective values for the features according to the respective attention weights for the features; and
processing, by the one or more computers, the embeddings to (i) identify, based on the embeddings, at least one of the relationships among the events that is omitted from the one or more records, and (ii) generate a prediction of a likelihood of a future outcome, wherein generating the prediction comprises:
generating, from the respective embeddings, a representation that aggregates information from multiple of the embeddings; and
processing the representation using a second neural network to generate the prediction of the likelihood of the future outcome, wherein the self-attention neural network and the second neural network have been trained jointly to minimize a loss function on training data that comprises a plurality of training examples.

2-4. (canceled)

5. The method of claim 1, wherein the prediction comprises a prediction of a current condition of the individual based on the one or more records.

6. The method of claim 1, wherein the prediction comprises a prediction of a likelihood of an undesirable outcome occurring to the individual.

7. (canceled)

8. The method of claim 1, wherein the mask value assigns a weight of negative infinity to the other features that are not connected to the feature in the vocabulary data.

9. The method of claim 1, wherein the vocabulary data specifies that features of a first type are only connected to features of a second type, but not to other features of the first type.

10. The method of claim 1, wherein the block inputs to a first self-attention block in the sequence are the features from the one or more records.

11. The method of claim 1, further comprising:
obtaining conditional probability data that specifies, for one or more first feature-second feature pairs, conditional probabilities of a first event represented by the first feature occurring in a particular set of one or more records given that a second event represented by the second feature occurred in the particular set of one or more records, wherein the self-attention for a first self-attention block in the sequence uses attention weights that are computed using the conditional probability data.

12. The method of claim 1, wherein training of the self-attention blocks is regularized to penalize attention weights of each self-attention block from deviating from attention weights generated by the preceding self-attention block.

13. A system comprising one or more computers and one or more storage devices storing instructions that when executed by the one or more computers cause the one or more computers to perform operations comprising:
obtaining, by the one or more computers, a plurality of features from one or more records associated with an individual, wherein each feature represents a corresponding event indicated by the one or more records and each of the plurality of features belongs to a vocabulary of multiple features that each represent a different event, and wherein relationships exist among at least some of the events indicated by the one or more records and the one or more records omit one or more of the relationships among the events;
maintaining, by the one or more computers, vocabulary data that specifies, for each of the features in the vocabulary, respective connections between the feature and one or more other features in the vocabulary;
generating respective embeddings for each of the features from the one or more records by applying a self-attention neural network that comprises a sequence of one or more self-attention blocks to the features from the one or more records, wherein each of the one or more self-attention blocks receives a respective block input for each of the features and applies self-attention over the block inputs to generate a respective block output for each of the features, and wherein, for one or more of the self-attention blocks, applying self-attention over the block inputs comprises:
generating, from the block inputs, a respective query for each feature by applying a first, learned linear transformation to the block input for the feature;
generating a respective key for each feature by applying a second, learned linear transformation to the block input for the feature;
generating a respective value for each feature by applying a third, learned linear transformation to the block input for the feature;
determining, for each feature, a respective attention weight for each of the plurality of features, wherein:
(i) for each other feature that is not connected to the feature in the vocabulary data, the respective attention weight is based on a respective similarity between the query for the feature and the key for the other feature and on a mask value that decreases an impact of the other feature on the feature, and
(ii) for each other feature that is connected to the feature in the vocabulary, the respective attention weight is based on a respective similarity between the query for the feature and the key for the other feature; and
combining, for each feature, the respective values for the features according to the respective attention weights for the features; and
processing the embeddings to (i) identify, based on the embeddings, at least one of the relationships among the events that is omitted from the one or more records, and (ii) generate a prediction of a likelihood of a future outcome, wherein generating the prediction comprises:
generating, from the respective embeddings, a representation that aggregates information from multiple of the embeddings; and
processing the representation using a second neural network to generate the prediction of the likelihood of the future outcome, wherein the self-attention neural network and the second neural network have been trained jointly to minimize a loss function on training data that comprises a plurality of training examples.

14-16. (canceled)

17. The system of claim 13, wherein the prediction comprises a prediction of a current condition of the individual based on the one or more records.

18. The system of claim 13, wherein the prediction comprises a prediction of a likelihood of an unfavorable outcome occurring to the individual.

19. (canceled)

20. One or more non-transitory computer-readable storage media storing instructions that when executed by one or more computers cause the one or more computers to perform operations comprising:
obtaining, by the one or more computers, a plurality of features from one or more records associated with an individual, wherein each feature represents a corresponding event indicated by the one or more records and each of the plurality of features belongs to a vocabulary of multiple features that each represent a different event, and wherein relationships exist among at least some of the events indicated by the one or more records and the one or more records omit one or more of the relationships among the events;
maintaining, by the one or more computers, vocabulary data that specifies, for each of the features in the vocabulary, respective connections between the feature and one or more other features in the vocabulary;
generating respective embeddings for each of the features from the one or more records by applying a self-attention neural network that comprises a sequence of one or more self-attention blocks to the features from the one or more records, wherein each of the one or more self-attention blocks receives a respective block input for each of the features and applies self-attention over the block inputs to generate a respective block output for each of the features, and wherein, for one or more of the self-attention blocks, applying self-attention over the block inputs comprises:
generating, from the block inputs, a respective query for each feature by applying a first, learned linear transformation to the block input for the feature;
generating a respective key for each feature by applying a second, learned linear transformation to the block input for the feature;
generating a respective value for each feature by applying a third, learned linear transformation to the block input for the feature;
determining, for each feature, a respective attention weight for each of the plurality of features, wherein:
(i) for each other feature that is not connected to the feature in the vocabulary data, the respective attention weight is based on a respective similarity between the query for the feature and the key for the other feature and on a mask value that decreases an impact of the other feature on the feature, and
(ii) for each other feature that is connected to the feature in the vocabulary, the respective attention weight is based on a respective similarity between the query for the feature and the key for the other feature; and
combining, for each feature, the respective values for the features according to the respective attention weights for the features; and
processing the embeddings to (i) identify, based on the embeddings, at least one of the relationships among the events that is omitted from the one or more records, and (ii) generate a prediction of a likelihood of a future outcome, wherein generating the prediction comprises:
generating, from the respective embeddings, a representation that aggregates information from multiple of the embeddings; and
processing the representation using a second neural network to generate the prediction of the likelihood of the future outcome, wherein the self-attention neural network and the second neural network have been trained jointly to minimize a loss function on training data that comprises a plurality of training examples.

21. The method of claim 1, wherein processing the respective embeddings includes predicting a graph structure that specifies the relationships among the events indicated by the one or more records, wherein the graph structure indicates relationships missing from the one or more records that indicate reflect dependencies among the events indicated by the one or more records.

22. The method of claim 1, wherein processing the respective embeddings includes predicting, based on the embeddings, one or more causal relationships among the events, wherein the one or more causal relationships were omitted from the one or more records.
